(19) United States
(12) Patent Application Publication
(10) Pub. No.: US 2023/0330158 A1
(43) Pub. Date: Oct. 19, 2023

(54) INTESTINAL MICROBIOTA BACTERIA AND COMPOSITION CONTAINING SAME FOR USE IN THE PREVENTION AND/OR TREATMENT OF DISEASES CHARACTERISED BY EXCESS 2-HYDROXYGLUTARATE

(71) Applicant: YSOPIA BIOSCIENCES, BORDEAUX CEDEX (FR)

(72) Inventors: Georges RAWADI, PARIS (FR); Sandrine CLAUS, BORDEAUX (FR); Laure RINALDI, BORDEAUX (FR); Katy, Nicole LECORF, MERIGNAC (FR)

(21) Appl. No.: 17/915,610

(22) PCT Filed: Jun. 24, 2020
(86) PCT No.: PCT/EP2020/067637

§ 371 Date: Sep. 29, 2022

(30) Foreign Application Priority Data
Jun. 25, 2019 (FR) FR1906845

(51) Int. Cl. A61K 35/741; A61K 9/19; A61P 35/00

(52) U.S. Cl. CPC A61K 35/741; A61K 9/19; A61P 35/00; A61K 2035/115

(57) ABSTRACT

The subject matter of the invention is a human intestinal microbiota bacterium selected from bacteria in the Christensenellaceae family, bacteria of the genus Negativibacillus and bacteria of the genus Massiliomicrobiota, or a composition containing same, for use in the prevention and/or treatment of diseases characterized by excess 2-hydroxyglutarate in humans or animals.

### TECHNICAL FIELD

The invention relates to the treatment of pathologies characterized by excess 2-hydroxyglutarate in humans or animals, in particular diseases having a degenerative effect on cells such as neurons, motor nerve cells, brain, colon, kidney, blood, skin, liver, lymph, prostate, thyroid, stomach, breast, pancreas, pituitary cancers, and 2-hydroxyglutaric aciduria.

### PRIOR ART

Diseases having a degenerative effect on neurons are disabling chronic diseases with a slow and discreet evolution that are characterized by the progressive loss of neurons in more or less localized regions of the nervous system, leading to cognitive, motor or perceptual complications. Ultimately, they can lead to death.

Indeed, neurons are cells that do not divide and do not renew themselves. Thus, neuron damage or death leads to their permanent disappearance from the human or animal body. However, the progressive degeneration and death of nerve cells are at the origin of problems related to movement (called ataxia) or to mental functioning (called dementia), which are characteristics of neurodegenerative diseases.

Diseases with a degenerative effect on neurons are generally related to aging and often affect people over 65 years of age. Known in particular are Alzheimer's disease, dementia, Parkinson's disease and associated disorders, prion diseases, neuromuscular diseases, Huntington's disease, spinocerebellar ataxia, progressive spinal muscular atrophy and amyotrophic lateral sclerosis.

Dementia is the most serious form of pathological brain aging. It causes an increasing impairment of memory and cognitive functions as well as behavioral disorders leading to a progressive loss of autonomy. It causes disabilities and dependency in the elderly.

Alzheimer's disease corresponds to a decline in cognition and memory. Nerve cells are gradually being destroyed in regions of the brain related to memory and language. Over time, the affected person has more and more difficulty memorizing events, recognizing objects and faces, remembering the meaning of words and exercising judgment.

Currently, the treatments offered consist in treating some of the patient's symptoms with drugs (Donepezil, Rivastigmine, Galantamine, Memantine) or in taking care of the patient in a non-pharmacological way (speech therapy, physiotherapy, osteopathy, psychology, occupational therapy, psychomotricity. These treatments are not satisfactory because they do not prevent the progression of the disease and treat certain disorders that are only intermittent.

Parkinson's disease affects the central nervous system, responsible for progressive disorders such as slowed movements, tremors, rigidity and cognitive disorders. It is the second most common neurodegenerative disease, after Alzheimer's disease.

Currently, the treatments offered consist of:
drug treatments to compensate for the lack of dopamine by mimicking the action of dopamine, by administering a substance that will be transformed into dopamine, by giving a substance that blocks the breakdown of dopamine,
surgical treatments consisting in stimulating deep brain function (implantation of electrodes in the brain). These treatments are not satisfactory because they have many side effects (nausea, vomiting, dyskinesia, behavioral disorders such as new addictions) and they improve the patient's quality of life but do not stop the progression of the disease.

Huntington's disease is a hereditary disease that is characterized by significant motor, cognitive and psychiatric disorders, and progresses until loss of autonomy and finally death. These symptoms can be classified into three major families.

Currently, the treatments offered consist in treating the symptoms to relieve the patient and slow down his physical and psychological deterioration with psychotropic drugs, neuroleptic drugs and rehabilitation through physiotherapy and speech therapy. These treatments are not satisfactory because they do not treat the disease curatively.

Prion diseases are diseases characterized in particular by a degeneration of the central nervous system. They are also called transmissible subacute spongiform encephalopathies (TSSEs). These diseases are caused by the accumulation in the brain of a normal, but malformed protein, the prion protein. These diseases are characterized by a rapid and fatal progression. The best known is Creutzfeldt-Jakob disease (CJD).

There is currently no treatment.

Neuromuscular diseases are diseases that affect the motor nerve cells of the spinal cord or motor neurons (spinal muscular atrophy, amyotrophic lateral sclerosis), the roots and nerves of the limbs (peripheral neuropathies), the junction between the nerve and the muscle (myasthenia) and the muscle (myopathies). They can affect the motor skills of the legs or arms, but sometimes also other organs and functions that depend on the muscles (motor skills of the eyes, speech, swallowing, digestion, breathing, the heart). The cause can be genetic or due to a malfunction of the immune system (“autoimmune” disease) that will cause damage to the nerves (dysimmune neuropathies) or the neuromuscular junction (myasthenia) or inflammation of the muscles (myositis). There are other possible causes: drug or environmental toxicity, vitamin deficiency, endocrine or general diseases, infections.

The treatments are different depending on the cause of the disease. In genetic diseases, therapeutic trials are in their early stages, and no routine treatment exists. When there is an anomaly in the cell metabolism, there are often drugs that aim to alleviate the consequences of this deficiency. In dysimmune diseases, there are many treatments that may be effective. However, undesirable side effects are known, such as hematologic effects.

Spinocerebellar ataxia corresponds to an autosomal dominant cerebellar ataxia type 1. It is characterized by ataxia, progressive external ophthalmoplegia and other neurological manifestations.

Currently, the treatments offered consist in directly treating the cause of the disease through surgery, blood-thinning drugs, antibiotics or steroids. Orphan drug treatments are also used. These treatments are not satisfactory because they are orphan drugs not developed for these indications, and the side effects can be varied: eosinophilia, leukopenia, thrombocytopenia, diarrhea, skin rash and increased liver enzymes. The other treatments can have the undesirable effect of bleeding, digestive disorders such as nausea, vomiting, diarrhea, allergies, photosensitization.

Spinal muscular atrophies or anterior spinal muscular atrophies are a group of neuromuscular diseases characterized by progressive muscle weakness due to degeneration and loss of anterior motor neurons in the spinal cord and brainstem nuclei. These diseases are presented in four forms depending on the age of onset and the severity of the disease. Currently, the treatments offered consist of a drug treatment (Nusinersen, known as Spinraza®), which is not effective in all forms of these diseases and has adverse respiratory and infectious effects.

Amyotrophic lateral sclerosis, or Charcot, is a disease characterized by progressive degeneration of motor neurons in the cerebral cortex. It causes progressive paralysis of the entire skeletal musculature of the limbs, the torso and the cephalic end.

Only one drug is currently offered to treat amyotrophic lateral sclerosis. It is Riluzole®, but this drug is not effective, does not improve the quality of life and prolongs survival by only a few months.

All diseases with a degenerative effect on neurons are characterized by excess production of 2-hydroxyglutarate by the sick person or animal (Gibson, K₂. M., Craigen, W., Herman, G. E. & Jakobs, C. D-2-Hydroxyglutaric Aciduria in a Newborn with Neurological Abnormalities: A New Neurometabolic Disorder? J. Inher. Metab. Dis 16, (1993); Ma, S. et al. L2hgdh Deficiency Accumulates I-2-Hydroxyglutarate with Progressive Leukoencephalopathy and Neurodegeneration. Mol. Cell. Biol. (2017). doi:10.1128/MCB.00492-16).

Accumulation of 2-hydroxyglutarate causes abnormal myelination, disrupts neuronal stem cell homeostasis and increases central nervous system cell mortality.

The various brain cancers are also characterized by excess 2-hydroxyglutarate production in the sick person or animal. Indeed, 2-hydroxyglutarate is known for its status as a marker for neurodegenerative diseases as well as its role as an oncometabolite in brain tumor development. 2-Hydroxyglutarate has a DNA repair inhibiting action. The production of high levels of 2-hydroxyglutarate results in the inhibition of DNA repair pathways in cancer cells, and therefore the accumulation of damaged DNA. 2-Hydroxyglutarate is also known for its role as a marker of renal cell carcinomas and in tissues under oxygen limitation or hypoxia conditions such as hepatocellular carcinomas or colon carcinomas; it thus has a role in kidney and colon cancers.

Inhibition of DNA repair by 2-hydroxyglutarate has been reported in other types of cancer: blood, skin, liver, lymph, prostate, thyroid, stomach, breast, pancreas, pituitary (Ye, D., Guan, K.-L. & Xiong, Y. Metabolism, Activity, and Targeting of D- and L-2-Hydroxyglutarates. Trends in cancer 4, 151-165 (2018)). 2-Hydroxyglutaric aciduria is a group of neurometabolic diseases with a broad clinical spectrum ranging from severe neonatal manifestations to progressive forms, and asymptomatic cases, characterized biochemically by elevated levels of 2-hydroxyglutaric acid in plasma, cerebrospinal fluid and urine.

L-2-hydroxyglutaric aciduria is characterized by psychomotor retardation, cerebellar ataxia and epilepsy, and D-2-hydroxyglutaric aciduria (see this term) by variable metabolic, neurological and dysmorphic manifestations.

Mutations in the L2HGDH (14q22.1) gene have been associated with L-2-hydroxyglutaric aciduria, and mutations in the D2HGDH (2q37.3) and IDH2 (15q26.1) genes have been associated with D-2-hydroxyglutaric aciduria.

There is no cure for diseases that have a degenerative effect on cells such as neurons and motor nerve cells, only interventions to improve the management of patients with diseases that have a degenerative effect on neurons and muscle nerve cells: early diagnosis, physical health optimization, cognitive and well-being activities, screening and treatment of physical and psychological comorbidities.

In addition, the main cancer treatments are chemotherapy and radiotherapy, which have limited effectiveness allowing a low survival rate and very heavy side effects such as hair loss, nausea and vomiting, diarrhea, decrease in white blood cells, red blood cells and platelets, lesions in the mouth, numbness or tingling sensations in the hands or feet, skin disorders and hand-foot syndrome, changes in the color and weakening of the nails, muscle and joint pain, menstrual cycle disorders, fatigue disorders, allergic reactions, damage to healthy cells by irradiation of healthy tissue next to the tumor, sexual disorders, fertility problems, inflammatory reaction, effects on blood cells. There is also no treatment for L- or D-2-hydroxyglutaric aciduria. In the case of D-2-hydroxyglutaric aciduria, epilepsy control is present; the prognosis depends entirely on the severity of the clinical picture and the progression of the disease, which cannot be predicted. In addition, the prognosis for L-2-hydroxyglutaric aciduria is poor for these patients, although most reach adulthood.

Thus, there is a great need for an effective treatment of both neurodegenerative diseases and cancers, in particular brain, kidney, colon and intestinal tumors, capable of acting on the synthesis of 2-hydroxyglutarate, that is easy to administer and that has no side effects.

### SUMMARY OF THE INVENTION

To meet this need, the invention targets the use of specific bacteria of the human intestinal microbiota, in particular bacteria of the Christensenellaceae family and/or bacteria of the Parasutterella genus and/or bacteria of the Negativibacillus genus and/or bacteria of the Massiliomicrobiota genus.

Bacteria of the family Christensenellaceae, including the genus Christensenella, have already been studied and described. This is the case in particular for Christensenella minuta, Christensenella massiliensis and Christensenella timonensis. Christensenella minuta in particular was described for the first time in 2012. In 2014, a study showed that it was the most heritable taxon in a cohort of British twins and that its presence is associated with a low body mass index. This correlation between Christensenella minuta and low body mass index was then observed in a dozen studies published since 2014 in geographically diverse populations. Finally, patent application US 2018/255819 describes the use of bacteria from the Christensenellaceae family in the treatment of obesity.

Bacteria of the Parasutterella genus have already been studied and described. This is particularly the case for Parasutterella excrementihominis and Parasutterella secunda, which have been observed in several studies, without causal explanation. Parasutterella excrementihominis was first described in 2009 and Parasutterella secunda was first described in 2011. Other species have also been studied. This is the case for Parasutterella mc1, which has been associated with the abundance of certain metabolites in healthy mice.

Bacteria of the Negativibacillus genus have been described. This is the case for Negativibacillus massiliensis, which was described in 2016. Bacteria of the Negativibacillus genus were characterized according to the description presented in “Negativibacillus massiliensis” gen. Nov., sp. Nov., isolated from human left colon, D. Ricaboni, M. Mailhe, V. Vitton, C. Andrieu, P.-E. Fournier, D. Raoult, New Microbe and New Infect 2017; 17: 36-38. No therapeutic use of bacteria of the Negativibacillus genus has ever been described or envisaged.

Bacteria of the Massiliomicrobiota genus have been studied little. Massiliomicrobiota timonensis and Massiliomicrobiota escudieri are known in particular.

Massiliomicrobiota timonensis was described for the first time in 2016. Massiliomicrobiota escudieri was described for the first time in 2018. Bacteria of the Massiliomicrobiota genus have been characterized according to the description presented in “Massiliomicrobiota timonensis,” a new bacterial species isolated from the human gut, S. Ndongo, S. Khelaifia, P.-E. Fournier, D. Raoult, New Microbe and New Infect 2016; 13: 25-26 and in “Massiliomicrobiota escudieri sp. nov. isolated as part of a culturomics exploration of the gut microbiota of renal cancer patients,” unpublished, Tidjani Alou, M., Derosa, L. and Zitvogel, L., submitted (11 Jun. 2018) U1015, Institut Gustave Roussy, 114 rue Edouard Vaillant, Villejuif 94800, France, Metropolitan. No therapeutic use of bacteria of the Massiliomicrobiota genus has ever been described or envisaged.

Surprisingly, and according to the invention:
bacteria of the Christensenellaceae family, in particular of the Christensenella genus, and in particular Christensenella minuta, Christensenella massiliensis and Christensenella timonensis,
bacteria of the Parasutterella genus, in particular Parasutterella excrementihominis and Parasutterella secunda,
bacteria of the Negativibacillus genus, in particular Negativibacillus massiliensis,
bacteria of the Massiliomicrobiota genus, in particular Massiliomicrobiota timonensis and Massiliomicrobiota escudieri, and
mixtures of at least two of these bacteria,
   when administered to humans or animals, are capable of acting on 2-hydroxyglutarate, the cause of neurodegenerative diseases and cancers.

This is why the subject of the invention is a bacterium of the Christensenellaceae family and/or bacteria of the Parasutterella genus and/or bacteria of the Negativibacillus genus and/or bacteria of the Massiliomicrobiota genus, for its use in the prevention and/or the treatment of at least one disease characterized by excess 2-hydroxyglutarate in humans or animals, in particular neurodegenerative diseases and cancers.

Advantageously, such bacteria, when they are administered to a human or an animal exhibiting a neurodegenerative disease or cancer, are capable of acting on the excess 2-hydroxyglutarate produced in these diseases.

For its use in the prevention or treatment of diseases characterized by excess 2-hydroxyglutarate in humans or animals, in particular in the prevention or treatment of neurodegenerative diseases and/or cancers, the bacteria of the Christensenellaceae family and/or bacteria of the Parasutterella genus and/or bacteria of the Negativibacillus genus and/or bacteria of the Massiliomicrobiota genus are preferably used in compositions. The invention therefore also relates to compositions comprising at least one bacterium of the Christensenellaceae family, preferably of the Christensenella genus, and/or a bacterium of the Parasutterella genus and/or a bacterium of the Negativibacillus genus and/or a bacterium of the Massiliomicrobiota genus, for its use in the prevention or treatment of diseases characterized by excess 2-hydroxyglutarate in humans or animals, in particular in the prevention or treatment of neurodegenerative diseases and/or cancers.

Other features and advantages will become apparent from the detailed description of the invention that follows.

### BRIEF DESCRIPTION OF THE DRAWINGS

FIG. 1 demonstrates the antiproliferative effect of the supernatants of bacteria according to the invention, in particular 3 strains of C. minuta on a human colon adenocarcinoma cell line, the HTC-116 line, after 24 and 48 hours of treatment.

FIG. 2 demonstrates the antiproliferative effect of the supernatants of bacteria according to the invention, in particular 3 strains of C. minuta on a human hepatocarcinoma cell line, the HepG2 line, after 24 and 48 hours of treatment.

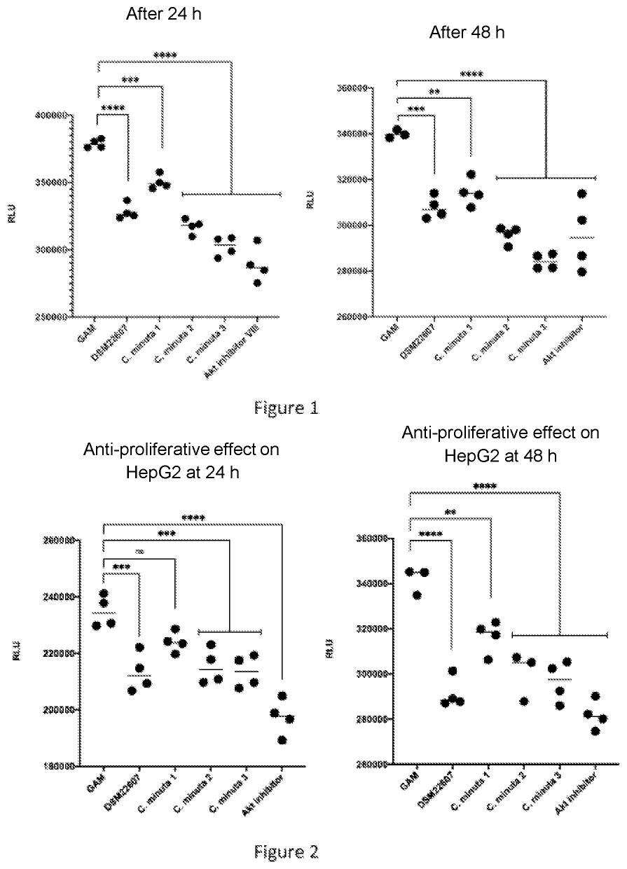

### DETAILED DESCRIPTION OF THE INVENTION

### Definitions

Within the meaning of the invention, “excess” or “hyperproduction” of 2-hydroxyglutarate means an excessive production of 2-hydroxyglutarate relative to the production in a healthy person or animal without pathology.

Within the meaning of the invention, “disease characterized by excess 2-hydroxyglutarate” or “disease characterized by hyperproduction of 2-hydroxyglutarate” means a disease whereof at least one cause is the excess or hyperproduction of 2-hydroxyglutarate in the body of the sick person or animal. This may in particular include a neurodegenerative disease or cancer.

Within the meaning of the invention, “marker” of a disease means a molecule or a substance whose assay makes it possible to follow the evolution of said disease.

Bacteria According to the Invention

The invention therefore relates to a bacterium of the Christensenellaceae family and/or bacteria of the Parasutterella genus and/or bacteria of the Negativibacillus genus and/or bacteria of the Massiliomicrobiota genus, for its use in the prevention and/or the treatment of at least one disease characterized by excess 2-hydroxyglutarate in humans or animals.

Bacteria of the Negativibacillus genus have been characterized according to the description presented in “Negativibacillus massiliensis” gen. Nov., sp. Nov., isolated from human left colon, D. Ricaboni, M. Mailhe, V. Vitton, C. Andrieu, P.-E. Fournier, D. Raoult, New Microbe and New Infect 2017; 17: 36-38 and in “Massiliomicrobiota escudieri sp. nov. isolated as part of a culturomics exploration of the gut microbiota of renal cancer patients,” unpublished, Tidjani Alou, M., Derosa, L. and Zitvogel, L., submitted (11 Jun. 2018) U1015, Institut Gustave Roussy, 114 rue Edouard Vaillant, Villejuif 94800, France, Metropolitan.

Bacteria of the Massiliomicrobiota genus have been characterized according to the description presented in “Massiliomicrobiota timonensis,” a new bacterial species isolated from the human gut, S. Ndongo, S. Khelaifia, P.-E. Fournier, D. Raoult, New Microbe and New Infect 2016; 13: 25-26.

In particular, the invention relates to a bacterium of the Christensenellaceae family, in particular of the Christensenella genus, and/or bacteria of the Parasutterella genus and/or bacteria of the Negativibacillus genus and/or bacteria of the Massiliomicrobiota genus, for its use in the prevention and/or the treatment of at least one neurodegenerative disease and/or of at least one cancer in humans or animals.

According to a variant, the invention relates to a bacterium of the Christensenellaceae family and/or bacteria of the Parasutterella genus and/or bacteria of the Negativibacillus genus and/or bacteria of the Massiliomicrobiota genus, for its use in the prevention and/or the treatment of at least one neurodegenerative disease chosen from Alzheimer's disease, dementia, Parkinson's disease and associated disorders, prion diseases, neuromuscular diseases, Huntington's disease, spinocerebellar ataxia, progressive spinal muscular atrophy and amyotrophic lateral sclerosis.

According to another variant, the invention relates to a bacterium of the Christensenellaceae family and/or bacteria of the Parasutterella genus and/or bacteria of the Negativibacillus genus and/or bacteria of the Massiliomicrobiota genus, for its use in the prevention and/or the treatment of at least one brain and/or kidney and/or liver and/or colon cancer, in humans or animals.

Preferably, the invention relates to a bacterium of the Christensenellaceae family and/or bacteria of the Negativibacillus genus and/or bacteria of the Massiliomicrobiota genus, for its use in the prevention and/or the treatment of at least one brain and/or kidney and/or liver and/or colon cancer, in humans or animals.

According to another variant, the invention relates to a bacterium of the Parasutterella genus, for its use in the prevention and/or treatment of at least one neurodegenerative disease, preferably chosen from Alzheimer's disease, dementia, Parkinson's disease and related disorders, prion diseases, neuromuscular diseases, Huntington's disease, spinocerebellar ataxia, progressive spinal muscular atrophy and amyotrophic lateral sclerosis.

According to the invention, the bacteria of the Christensenellaceae family and/or the bacteria of the Parasutterella genus and/or the bacteria of the Negativibacillus genus and/or the bacteria of the Massiliomicrobiota genus, when they are administered to a human or an animal presenting a neurodegenerative disease, are capable of acting on the excess molecules produced in a neurodegenerative disease or cancer, in particular on 2-hydroxyglutarate.

In diseases with a degenerative effect on neurons, the decrease in the amount of 2-hydroxyglutarate is a sign of the reduction in these diseases with a degenerative effect on neurons, that is to say, the bacteria at the origin of this production are less stimulated. From then on, the excessive production responsible for the disease having a degenerative effect is slowed down and the system gradually returns to normal. In particular, a substrate of 2-hydroxyglutarate, glutarate, decreases when 2-hydroxyglutarate increases. Thus, a return to normal through a decrease in 2-hydroxyglutarate is accompanied by an increase in glutarate.

In cancers, the decrease in the synthesis of 2-hydroxyglutarate is the sign of reduced inhibition of the DNA repair pathways, that is to say, the bacteria at the origin of this production are less stimulated. From then on, the excessive production responsible for DNA repair inhibition is slowed down and the system gradually returns to normal. In particular, a substrate of 2-hydroxyglutarate, glutarate, decreases when 2-hydroxyglutarate increases. Thus, a return to normal through a decrease in 2-hydroxyglutarate is accompanied by an increase in glutarate.

In 2-hydroxyglutaric aciduria, the reduction in 2-hydroxyglutarate synthesis is the sign of the reduction in the clinical symptoms associated with this disease. In particular, a substrate of 2-hydroxyglutarate, glutarate, decreases when 2-hydroxyglutarate increases. Thus, a decrease in 2-hydroxyglutarate is accompanied by an increase in glutarate.

Where the useful bacteria according to the invention are administered to humans or animals in an effective quantity for an action on 2-hydroxyglutarate, that is to say, to reduce its production in the body. According to a suitable embodiment, the bacterium or bacteria can be administered at a dose of 10⁹ to 10¹² colony-forming units (CFU) per day, regardless of the weight of the person or animal. It is preferably a single dose, i.e., administered once daily, or a dose before each meal (three times a day).

The useful bacterium or bacteria according to the invention are:
bacteria of the Christensenellaceae family, preferably of the genus Christensenella. It may be, in particular, Christensenella massiliensis, Christensenella timonensis and/or Christensenella minuta. According to a particularly suitable variant, it is Christensenella minuta. These bacteria can be isolated from human stools for example according to the protocols published by Morotomi et al., 2012 (Morotomi, M., Nagai, F. & Watanabe, Y. Description of Christensenella minuta gen. nov., sp. nov., isolated from human faeces, which forms a distinct branch in the order Clostridiales, and proposal of Christensenellaceae fam nov. INTERNATIONAL JOURNAL OF SYSTEMATIC AND EVOLUTIONARY MICROBIOLOGY 62, 144-149 (2012)) and NDongo et al., 2016 (Ndongo, S., Dubourg, G., Khelaifia, S., Fournier, P. E. & Raoult, D. Christensenella timonensis, a new bacterial species isolated from the human gut. New Microbes and New Infections 13, 32-33 (2016)). These documents also describe the culture methods of the useful bacteria according to the invention.
bacteria of the Parasutterella genus. It may in particular be Parasutterella excrementihominis and Parasutterella secunda. These bacteria can be isolated from human stool for example according to the protocols published by Nagai et al. 2009 (Parasutterella excrementihominis gen. nov., sp. nov., a member of the family Alcaligenaceae isolated from human faeces Nagai F Morotomi M Sakon H Tanaka R, International Journal of Systematic and Evolutionary Microbiology, 2009) and Morotomi et al. 2011 (Parasutterella secunda sp. nov., isolated from human faeces and proposal of Sutterellaceae fam. nov. in the order Burkholderiales, Morotomi M Nagai F Watanabe Y, International Journal of Systematic and Evolutionary Microbiology, 2011). These documents also describe the culture methods of the useful bacteria according to the invention.
bacteria of the Negativibacillus genus, preferably Negativibacillus massiliensis. These bacteria can be isolated from human stool for example according to the protocols published by Fournier et al. 2016 (“Negativibacillus massiliensis” gen. nov., sp. nov., isolated from human left colon, Fournier P Ricaboni D Vitton V Raoult D Andrieu C Mailhe M, New Microbes and New Infections, 2016). This document also describes the culture methods of the useful bacteria according to the invention.
bacteria of the genus Massiliomicrobiota. It may in particular be Massiliomicrobiota timonensis and Massiliomicrobiota escudieri. According to a particularly suitable variant, it is Massiliomicrobiota timonensis. These bacteria can be isolated from human stool for example according to the protocol published by Ndongo et al. 2016 (“Massiliomicrobiota timonensis,” a new bacterial species isolated from the human gut. Ndongo S Khelaifia S Fournier P Raoult D. New microbes and new infections, 2016 vol: 13 pp: 25-6). New Microbes and New Infections 13, 32-33 (2016)). This document also describes the culture methods of the useful bacteria according to the invention.
and mixtures of at least two of these bacteria.

The useful bacterium or bacteria according to the invention, for their previously described use, are preferably administered in a composition.

Composition Comprising Bacteria According to the Invention

The invention therefore also relates to a composition comprising at least one bacterium of the Christensenellaceae family and/or a bacterium of the Parasutterella genus and/or a bacterium of the Negativibacillus genus and/or a bacterium of the Massiliomicrobiota genus, in the prevention and/or treatment of diseases characterized by excess 2-hydroxyglutarate, in particular neurodegenerative diseases and/or cancers, in humans or animals. The bacterium or bacteria are present in an effective amount in the composition, allowing an effect on the 2-hydroxyglutarate and on the disease(s) to be treated, in particular the neurogenerative diseases and/or cancers, of the treated persons or animals.

Preferably, the useful composition according to the invention comprises 10⁶ to 10¹² colony-forming units (CFU) of bacteria of the Christensenellaceae family and/or of bacteria of the Parasutterella genus and/or of bacteria of the Negativibacillus genus and/or of bacteria of the Massiliomicrobiota genus per daily dose of composition to be administered. Preferably, this corresponds to a daily dose of bacteria to be administered, regardless of the weight of the person or the animal. Preferably, this dose is administered once per day.

The useful composition according to the invention may be in liquid form. It may in particular comprise bacteria of the Christensenellaceae family and/or bacteria of the Parasutterella genus and/or bacteria of the Negativibacillus genus and/or bacteria of the Massiliomicrobiota genus and a culture medium for said bacteria that makes it possible to preserve them. This medium may be, for example, Columbia agar medium for bacteria of the Christensenellaceae family, bacteria of the Negativibacillus genus, bacteria of the Massiliomicrobiota genus, or BTU medium for bacteria of the Parasutterella genus, anaerobic enriched with sheep blood, or an equivalent medium containing no animal byproduct.

According to one variant, the useful composition according to the invention may be in solid form. In this case, the bacteria may be present in freeze-dried form, and may also comprise excipients such as, for example, microcrystalline cellulose, lactose, sucrose, fructose, levulose, starches, stachyose, raffinose, amylum, calcium lactate, magnesium sulphate, sodium citrate, calcium stearate, polyvinylpyrrolidone, maltodextrin, galactooligosaccharides, fructooligosaccharides, pectins, beta-glucans, lactoglobulins, isomaltooligosaccharides, polydextroses, sorbitol and/or glycerol.

The useful compositions according to the invention may in particular be in the form of powder, microencapsulated powder, gelcap, capsule, tablet, lozenge, granules, emulsion, suspension or suppository. According to a particularly suitable embodiment, they may be in a gastro-resistant form, such as a coated tablet containing microencapsulated bacteria.

When the compositions are in solid form, they are preferably packaged in capsules or in a coating hermetically sealed against light and oxygen, maintained at an ambient temperature of between 15° C. and 40° C. and a humidity level between 3% and 70%.

The bacteria can be used alive, or inactivated, for example by heat, exposure to an appropriate pH, gamma radiation or high pressure.

They can all be alive or all inactivated.

Preferably, at least part of the bacteria is made up of live bacteria, in particular at least 50% (by number), even more preferably at least 90% (by number).

Thus, according to a suitable embodiment, the bacteria present in the useful composition according to the invention are at least 50% living bacteria (by number), preferably at least 90% living bacteria (by number), and even more preferentially all living.

The useful bacteria according to the invention, and in particular the compositions that include them, can be administered orally, topically, through the lungs (inhalation) or rectally.

The useful compositions according to the invention, in addition to the useful bacteria according to the invention, can comprise other compounds, such as:
at least one probiotic, and/or
at least one bacterium making it possible to create an anaerobic environment favorable to the bacteria present in the composition, such as at least one bacterium chosen from bacteria of the genus Lactobacillus spp., Bifidobacterium spp., Streptococcus spp. and/or at least one other organism promoting the anaerobic conditions necessary for the survival of Christensenellaceae, such as at least one yeast chosen from Saccharomyces spp. or microorganisms of the Methanobacteriaceae family, and/or
at least one bacterium associated with the ecosystem of the bacteria present in the composition, since they facilitate their survival in the intestine, such as at least one bacterium chosen from bacteria of the phylum Firmicutes, Bacteroidetes, Actinobacteria, Tenericutes, betaproteobacteria and Verrucomicrobia, and/or
at least one bacterium chosen from bacteria of the order Burkholderiales, Clostridales, Verrucomicrobiales, Aeromonadales, Alteromonadales, ML615J-28, RF32, YS2, of the family Clostridiaceae, Lachnospiraceae, Erysipelotrichaceae, Ruminococcaceae, Bacteroidaceae, Enterococcaceae, Rikenellaceae, Dehalobacteriaceae, Veillonellaceae, Lactobacillaceae and/or
at least one bacterium chosen from bacteria of the genus Faecalibacterium, Akkermansia, Eubacterium, Sutterella, Burkholderia, Derxia, Brackiella, Oligella, Pelistega, Taylorella, Tetrathiobacter, Advenella, Alcicaligenes, Pigmentiphaga, Kerstersia, Achromobacter, Bordetella, Castellaniella, Pusillimonas, Turicibacter and Oscillospira such as, for example, Faecalibacterium prausnitzii, Akkermansia muciniphila, Eubacterium halii, Oscillospira guilliermondii, Turicibacter sanguinis, Suterella parvirubra, Derxia gummosa, Brackiella oedipodis, Oligella urethralis, Pelistega europea, Taylorella equigenitalis, Tetrathiobacter kashmirensis, Advenella incenata, Alcaligenes faecalis, Pigmentiphaga kullae, Kerstersia gyiorum, Achromobacter xylosoxidans, Bordetella pertussis, Castellaniella defragrans, Pusillimonas noertemannii and/or
at least one prebiotic such as for example at least one prebiotic chosen from galactooligosaccharides, fructooligosaccharides, inulins, arabinoxylans, beta-glucans, lactoglobulins and/or beta-caseins, and/or
at least one polyphenol such as for example at least one polyphenol chosen from quercetin, kaempferol, resveratrol, flavones (such as luteolin), flavan-3-ols (such as catechins), flavanones (such as naringenin), isoflavones, anthocyanidins, proanthocyanidins, and/or
at least one mineral and/or at least one vitamin and/or at least one nutritional agent, and/or
at least one active pharmaceutical ingredient, preferably at least one active pharmaceutical ingredient having a therapeutic effect on the pathology or pathologies (diseases having a degenerative effect on neurons or muscle nerve cells and/or cancer(s) in particular) for which the bacteria present in the composition are used, such as for example:

Drugs against the degenerative effect of neurons and muscle nerve cells Donepezil, Rivastigmine, Galantamine, Memantine, Riluzole, Nusinersen, psychotropics, thinners, neuroleptics

Chemotherapy drugs including targeted therapies or biotherapies, hormone therapy or immunotherapy

The invention is now illustrated by examples of useful bacteria according to the invention, methods for culturing these bacteria, examples of compositions containing them and test results demonstrating the effectiveness of bacteria of the Christensenellaceae family and/or bacteria of the Parasutterella genus and/or bacteria of the Negativibacillus genus and/or bacteria of the Massiliomicrobiota genus on 2-hydroxyglucarate and consequently on the diseases induced by an excess of this molecule.

### EXAMPLES

### Example 1: Christensenella minuta

The Christensenella minuta bacteria can be cultured according to the operating protocol described below.
1/ Dissolve a dehydrated RCM (“Reinforced Clostridial Medium”) medium in distilled water
2/ Add 0.5 mL/L of resazurin-Na solution (0.1% w/v)
3/ Bring to a boil and cool to room temperature while injecting a gaseous mixture of 80% N₂ and 20% CO₂
4/ Spread the medium under the same gaseous atmosphere in anoxic Hungate-type tubes or in serum vials, then autoclave
5/ Before use, add 1.0 g of sodium carbonate per liter from a sterile anoxic stock solution prepared with a gaseous mixture of 80% N₂ and 20% CO₂
6/ Check the pH of the medium after autoclaving and adjust the pH between 7.3 and 7.5, using a sterile anoxic stock solution of sodium bicarbonate (5% w/v) prepared in a gaseous atmosphere at 80% N₂ and at 20% CO₂.

### Example 2: Christensenella massiliensis

The Christensenella massiliensis bacteria can be cultured according to the operating protocol described below.
1/ Prepare a carboxymethylcellulose (N₂/CO₂) medium by following the instructions below provided by DSMZ (Deutsche Sammlung von Mikroorganismen und Zellkulturen), presented in Table 1.

TABLE 1

| | | |
|---|---|---|
| Casitone | 30.0 | g |
| Yeast extract | 5.0 | g |
| K₂HPO₄ | 5.0 | g |
| Na-resazurin solution (0.1% w/v) | 0.5 | mL |
| L-Cysteine-HCl × H₂O | 0.5 | g |
| D-Glucose | 4.0 | g |
| Cellobiose | 1.0 | g |
| Maltose | 1.0 | g |
| Na₂CO₃ | 1.0 | g |
| Meat filtrate (see Table 2) | 1000 | mL |

2/ Dissolve the different constituents listed in the table above, except cysteine, carbohydrates and carbonate.
3/ Boil the medium for 1 min, then let it cool to room temperature under a gaseous atmosphere containing 80% N₂ and 20% CO₂.
4/ Add 0.5 g/L of L-cysteine-HCl x H₂O and pour it under the same gaseous atmosphere into Hungate-type tubes (for strains requiring meat particles, introduce these first into the tube; use 1 part meat particles to 4 or 5 parts liquid).
5/ Autoclave at 121° C. for 20 min.
6/ After autoclaving, add glucose, cellobiose, maltose and starch from sterile anoxic stock solutions prepared with 100% N₂ gas and carbonate from a sterile anoxic stock solution prepared under gaseous mixtures at 80% N₂ and 20% CO₂.
7/ Adjust the pH of the medium to 7, if necessary.

The composition of the meat filtrate is shown in Table 2.

TABLE 2

| | | |
|---|---|---|
| Ground meat (no fat) | 500.0 | g |
| 1N NaOH | 25.0 | mL |
| Distilled water | 1000 | mL |

The meat filtrate is prepared as follows.
a/ Use lean beef or horse meat.
b/ Remove fat and connective tissue before chopping.
c/ Mix the meat, water and NaOH, then boil for 15 minutes with stirring.
d/ Allow to cool to room temperature, remove fat from the surface and filter, retaining meat particles and filtrate.
e/ Add water to the filtrate to a final volume of 1000.0 mL.

The bacteria must be grown under anaerobic conditions at 37° C.

### Example 3: Christensenella timonensis

The Christensenella timonensis bacteria can be cultivated according to the same procedure as that described in Example 2 for Christensenella massiliensis.

### Example 4: Parasutterella excrementihominis

The Parasutterella excrementihominis bacteria can be cultured according to the operating protocol described below.

BTU=(ground meat medium+Formate/fumarate)+5% bovine serum
Ground meat medium
Ground beef (fat free) (500.0 g)+distilled water (1000.0 mL)+1 N NaOH (25.0 mL).

Use lean beef or horse meat. Remove fat and connective tissue before chopping. Mix the meat, the water and the NaOH, then boil for 15 minutes with stirring. Cool to room temperature, remove fat from the surface and filter while keeping both: meat particles and filtrate. Add water to the filtrate for a final volume of 1000.0 mL, and then add: casitone (30.0 g), yeast extract (5.0 g), K₂HPO₄ (5.0 g), resazurin (1.0 mg)

Boil under a Nitrogen atmosphere, add 0.5 g/l of cystine and adjust the pH to 7.0. Distribute 7 mL of medium under anaerobic conditions in Hungate tubes containing the meat particles (use one part meat particles for 4 to 5 parts liquid). Autoclave at 121° C. for 30 min.

To prepare the agar: use test tubes and put 15 g of agar therein for 1000.0 L of medium.

In some cases, it is possible to add Haemin, Vitamin K1 or Vitamin K3 if needed.

Add 1000.0 mL of medium after autoclaving: Haemin solution (10.0 mL)+Vitaline K1 or K3 solution (0.2 mL).

Haemin solution: dissolve 50 mg of Haemin in 1 mL of 1 N NaOH, add 100 mL of distilled water and sterilize by filtration, store refrigerated.

Vitamin K1 solution: dissolve 0.1 mL of vitamin K1 in 20 mL of 95% ethanol and sterilize by filtration. Store refrigerated in an amber bottle.

Vitamin K3 solution:

Dissolve 5 mg/mL of vitamin K3 in 10 mL of 95% ethanol and sterilize by filtration.

Store refrigerated in an amber bottle.

Formate/fumarate solution

Mix Na-formate (6.0 g)+Na-fumarate (6.0 g)+distilled water (100.0 mL). Sterilize by filtration. Add 30 microliter per mL of medium 78 before inoculation.

### Example 5: Parasutterella secunda

The Parasutterella secunda bacteria can be cultured according to the operating protocol described below.

EG Medium:
Add 0.2 g of L-cystine to 50 mL of 1 N HCl and mix vigorously. Then add: 2.4 g of Lab-Lemco powder, 10.0 g of peptone proteose No3, 5.0 g of yeast extract, 4.0 g of Na₂HPO₄, 1.5 g of glucose, 0.5 g of soluble starch, 15.0 g of agar, 0.5 g of L-cysteine HCl H₂O, then distilled water to reach a volume of 950.0 mL.
Adjust the pH to 7.6-7.8.
Autoclave and cool to 50° C., then add 50.0 mL of horse blood aseptically.
Mix vigorously and distribute in appropriate sterile containers (Petri dishes or sterile tubes).

Or Columbia blood agar medium with 5% horse blood:
Prepare the Columbia blood agar base (Oxoid CM331) according to the instructions, sterilize and cool to 45° C.
add 50.0 mL of aseptically defibrinated horse blood.
mix and disperse quickly in appropriate sterile containers (Petri dishes or sterile tubes).

### Example 6: Negativibacillus massiliensis

The Negativibacillus massiliensis bacteria can be cultured according to the operating protocol described below.

Species obtained by growth on Columbia agar medium (bioMérieux, Marcy I'Etoile, France) with 5% sheep blood under anaerobic atmosphere (anaeroGEN, Oxoid, Dardilly, France) after 14 days of enrichment of a fresh colon sample left placed in a bottle cultured in blood (Becton Dickinson, Pont de Claix, France) with 5 mL of sheep blood (bioMérieux) and 5 mL of 0.2 μm filtered rumen (Thermo Fisher Scientific, Villebon-sur-Yvette, France) at 37° C. Then 5 days of anaerobic incubation on Columbia agar medium enriched with 5% sheep blood

### Example 7: Massiliomicrobiota timonensis

Massiliomicrobiota timonensis can be cultivated according to the operating protocol described as follows:
Columbia bioMérieux agar medium, Marcy I'Etoile, France) enriched with 5% Columbia agar sheep blood
at 37° C.
anaerobic atmosphere generated by AnaeroGen (bioMérieux).
72 hours of incubation

### Example 8: Massiliomicrobiota escudieri

The Massiliomicrobiota escudieri bacteria can be cultivated according to the same procedure as that described in Example 7 for Massiliomicrobiota timonensis.

### Example 9: Useful Composition of Christensenella minuta According to the Invention in Liquid Form

An example of a useful composition according to the invention in liquid form is a composition comprising Christensenella minuta 10⁹ CFU/mL in the RCM anaerobic culture medium described above, modified to contain no product of animal origin and enriched with 5% glycerol.

The composition of Example 9 was obtained from an RCB (“research cell bank”) prepared with Christensenella minuta 10¹⁰ CFU/mL and stored frozen at −20° C. in a bag hermetically sealed to oxygen.

The frozen composition must be warmed to room temperature until a liquid form is found before use.

### Example 10: Useful Composition of Christensenella massiliensis According to the Invention in Liquid Form

An example of a useful composition according to the invention in liquid form is a composition comprising Christensenella massiliensis 10⁹ CFU/mL in the carboxymethylcellulose anaerobic culture medium described above, modified to contain no product of animal origin and enriched with 5% glycerol.

The composition of Example 10 was obtained from an RCB (“research cell bank”) prepared with Christensenella massiliensis 10¹⁰ CFU/mL and stored frozen at −20° C. in a bag hermetically sealed to oxygen.

The frozen composition must be warmed to room temperature until a liquid form is found before use.

### Example 11: Useful Composition of Christensenella timonensis According to the Invention in Liquid Form

An example of a useful composition according to the invention in liquid form is a composition comprising Christensenella timonensis 10⁹ CFU/mL in the carboxymethylcellulose anaerobic culture medium described above, modified to contain no product of animal origin and enriched with 5% glycerol.

The composition of Example 11 was obtained from an RCB (“research cell bank”) prepared with Christensenella timonensis 10¹⁰ CFU/mL and stored frozen at −20° C. in a bag hermetically sealed to oxygen.

The frozen composition must be warmed to room temperature until a liquid form is found before use.

### Example 12: Useful Composition of Parasutterella excrementihominis According to the Invention in Liquid Form

An example of a useful composition according to the invention in liquid form is a composition comprising Parasutterella excrementihominis 10⁹ CFU/mL in the BTU anaerobic culture medium described above, modified to contain no product of animal origin and enriched with 5% glycerol.

The composition of Example 12 was obtained from an RCB (“research cell bank”) prepared with Parasutterella excrementihominis 10¹⁰ CFU/mL and stored frozen at −20° C. in a bag hermetically sealed to oxygen.

The frozen composition must be warmed to room temperature until a liquid form is found before use.

### Example 13: Useful Composition of Parasutterella secunda According to the Invention in Liquid Form

An example of a useful composition according to the invention in liquid form is a composition comprising Parasutterella secunda 10⁹ CFU/mL in the EG culture medium or the Columbia anaerobic culture medium described above, modified to contain no product of animal origin and enriched with 5% glycerol.

The composition of Example 13 was obtained from an RCB (“research cell bank”) prepared with Parasutterella secunda 10¹⁰ CFU/mL and stored frozen at −20° C. in a bag hermetically sealed to oxygen.

The frozen composition must be warmed to room temperature until a liquid form is found before use.

### Example 14: Useful Composition of Negativibacillus massiliensis According to the Invention in Liquid Form

An example of a useful composition according to the invention in liquid form is a composition comprising Negativibacillus massiliensis 10⁹ CFU/mL in the EG culture medium or the Columbia anaerobic culture medium described above, modified to contain no product of animal origin and enriched with 5% glycerol.

The composition of Example 14 was obtained from an RCB (“research cell bank”) prepared with Negativibacillus massiliensis 10¹⁰ CFU/mL and stored frozen at −20° C. in a bag hermetically sealed to oxygen.

The frozen composition must be warmed to room temperature until a liquid form is found before use.

### Example 15: Useful Composition of Massiliomicrobiota timonensis According to the Invention in Liquid Form

An example of a useful composition according to the invention in liquid form is a composition comprising Massiliomicrobiota timonensis 10⁹ CFU/mL in the EG culture medium or the Columbia anaerobic culture medium described above, modified to contain no product of animal origin and enriched with 5% glycerol.

The composition of Example 15 was obtained from an RCB (“research cell bank”) prepared with Massiliomicrobiota timonensis 10¹⁰ CFU/mL and stored frozen at −20° C. in a bag hermetically sealed to oxygen.

The frozen composition must be warmed to room temperature until a liquid form is found before use.

### Example 16: Useful Composition of Massiliomicrobiota Escudieri According to the Invention in Liquid Form

An example of a useful composition according to the invention in liquid form is a composition comprising Massiliomicrobiota escudieri 10⁹ CFU/mL in the EG culture medium or the Columbia anaerobic culture medium described above, modified to contain no product of animal origin and enriched with 5% glycerol.

The composition of Example 16 was obtained from an RCB (“research cell bank”) prepared with Massiliomicrobiota escudieri 10¹⁰ CFU/mL and stored frozen at −20° C. in a bag hermetically sealed to oxygen.

The frozen composition must be warmed to room temperature until a liquid form is found before use.

### Example 17: Composition of at Least One Useful Bacterium According to the Invention in Liquid Form Mixed with at Least One Other Distinct Useful Bacterium According to the Invention in Liquid Form

An example of a useful composition according to the invention in liquid form can be obtained by mixing at least one composition of Examples 9 to 16 with at least one distinct composition from Examples 9 to 16.

### Example 18: Useful Composition According to the Invention in Solid Form

An example of a useful composition according to the invention in freeze-dried form can be obtained by freeze-drying one of the compositions of Examples 9 to 17 in the frozen state.

In Vitro Demonstration of the Treatment Effect on Diseases Characterized by Excess 2-Hydroxyglucarate, in Particular Neurodegenerative Diseases and Cancers

The objective of this study is to demonstrate in vitro the treatment effect of bacteria according to the invention on neurodegenerative diseases and cancers. The demonstration was carried out on 2-hydroxyglutarate and its precursor, glutarate.

The procedure of the study is described below.
1/ Fermentation protocol using feces of human origin containing Christensenella spp., Parasutterella spp., Negativibacillus spp., Massiliomicrobiota spp.:
The donors must not have taken antibiotics during the six months preceding the experiment and have no history of gastrointestinal disorders. The donors were between 18 and 60 years old.
The collection of fresh samples of their feces is obtained in sterile plastic containers, stored in anaerobic bottles containing a 2.5 L sachet of AnaeroGen™ from Oxoid™ O₂<0.1%; CO₂: 7-15%). These samples were brought to the laboratory within two hours of their production.
Feces samples were diluted 1/5 (weight/volume) in phosphate buffered saline (1 M) (PBS), pH 7.4. The suspension was homogenized in a stomacher for 120 seconds.
Basic nutrient medium: the basic nutrient medium was prepared from 2 g/L tryptone soy broth, 2 g/L yeast extract, 0.1 g/L NaCl, 0.04 g/L K2HPO₄, 0.01 g/L MgSO₃·7H2O, 0.01 g/L CaCl₂·6H2O, 2 g/L NaHCO₃, 0.5 g/L L-cystine HCl, 2 mL/L tween 80, 10 μL/L vitamin K1, 0.05 g/L heme, 0.05 g/L bile salts, 4 ml/L resazarin (pH 7)
Fermentation in a biofermenter: The 20 mL capacity biofermenters contained 18 mL of autoclaved base nutrient medium (121° C. for 15 minutes) poured aseptically into the sterile biofermenters. This system was allowed to stand overnight with oxygen-free nitrogen bubbling through the medium at a rate of 2 mL/min. The pH was maintained between 6.7 and 6.9 using HCl or NaOH (0.5 M). The temperature of each biofermenter was controlled at 37° C. and the contents of the container were homogenized with a magnetic mixer
a mixture of predigested proteins (0.35 g) was added to the containers before inoculation with 2 mL of fecal inocula at TO. The predigested proteins were obtained according to the gastrointestinal digestion protocol adapted from that of Versantvoort et al (2005).
the samples were collected before fermentation (T0) and after 48 hours of fermentation (T48), and frozen at −80° C. until analysis.
2/ Quantification of 2-hydroxyglutarate and glutarate. 50 μL of samples collected and stored at −80° C. was mixed with 20 μL of Milli-Q water containing internal standards.
The mixture was mixed and filtered through a 5-kDa threshold filter to remove macromolecules.
The metabolites were detected by capillary electrophoresis-time-of-flight mass spectrometry (CE-TOFMS) analyses. The peak detection limit was determined based on the signal-to-noise ratio, S/N=3.

Relative peak area=(metabolite peak area)/(internal standard peak area×amount of sample).
3/ Quantification of Christensenella spp., Parasutterella spp., Negativibacillus spp., Massiliomicrobiota spp.
The DNA contained in the samples was extracted using the NucleoSpin®96 Soil kit from Macherey-Nagel according to the manufacturer's instructions.
The total extracted DNA was then randomly fragmented into 350 bp fragments and then used to build a library using the NEBNext Ultra II kit by New England Biolabs according to the manufacturer's instructions.
The library was then sequenced using 2×150 bp paired-end sequencing on an Illumina HiSeq platform.
The abundance of bacteria was measured by creating a metagenomic species catalog (MGS) from a reference catalog containing 22 M genes. These MGSs were then associated with an appropriate taxonomic level. In the case of Christensenella, these were detected at the genus level and are therefore referred to in this experiment as Christensenella spp.

The relative amount of 2-hydroxyglutarate and the relative abundance of Christensenella spp., Parasutterella spp., Negativibacillus spp., Massiliomicrobiota spp were analyzed and correlated, obtaining a linear regression of R=−0.42, R=−0.41, R=−0.31, R=−0.26 (n=18).

The relative amount of glutarate and the relative abundance of Christensenella spp., Parasutterella spp., Negativibacillus spp., Massiliomicrobiota spp were analyzed and correlated, obtaining a linear regression of R=0.41, R=0.43, R=0.51, R=0.36 (n=18).

The results are shown in Table 3.

TABLE 3

| | Relative | Relative | Relative | Relative | | |
|---|---|---|---|---|---|---|
| | abundance of | abundance of | abundance of | abundance of | Relative | Relative |
| | Christensenella | Parasutterella | Negativibacillus | Massiliomicrobiota | amount of | amount of |
| | spp | spp | spp | spp | 2-hydroxyglutarate | glutarate |
| Samples | (×10⁻³) | (×10⁻³) | (×10⁻²) | (×10⁻³) | (×10⁻⁵) | (×10⁻⁵) |
| V1 | 7.55 | 560.67 | 9.02 | 0 | 3.47 | 0 |
| V2 | 3.18 | 258.72 | 2.48 | 4.14 | 3.01 | 0 |
| V3 | 8.19 | 266.78 | 0 | 1.23 | 0 | 5.4332 |
| V4 | 2.6 | 1362.44 | 1.90 | 0 | 3.24 | 0 |
| V5 | 1.26 | 5.21 | 0 | 0 | 6.06 | 0 |
| V6 | 2.87 | 1163.56 | 0 | 0 | 2.91 | 0 |
| V7 | 7.20 | 7297.76 | 268.30 | 1.29 | 0 | 31.44 |
| V8 | 2.91 | 2456.31 | 45.18 | 1.19 | 0 | 22.301 |
| V9 | 6.32 | 7271.23 | 0 | 1.23 | 0 | 20.88 |
| V10 | 1.21 | 196.88 | 2.91 | 0 | 0 | 13.56 |
| V11 | 4.23 | 231.77 | 121.61 | 0 | 0 | 21.43 |
| V12 | 1.49 | 6342.36 | 0 | 0 | 0 | 19.68 |
| V13 | 9.83 | 4535.74 | 262.37 | 5.84 | 0 | 41.08 |
| V14 | 4.12 | 2517.99 | 13.96 | 4.31 | 0 | 20.72 |
| V15 | 6.45 | 4798.48 | 0 | 4.08 | 0 | 22.16 |
| V16 | 2.02 | 544.48 | 8.85 | 0 | 3.31 | 22.54 |
| V17 | 5.23 | 111.85 | 606.77 | 0 | 0 | 24.81 |
| V18 | 7.57 | 17842.41 | 0 | 0 | 0 | 22.32 |

A negative correlation is observed between the bacteria according to the invention and 2-hydroxyglutarate as well as a positive correlation between the bacteria according to the invention and glutarate, which demonstrates a protective effect of bacteria of the Massiliomicrobiota genus against neurodegenerative diseases and cancers.

Thus, the bacteria according to the invention are able to act by decreasing the production of 2-hydroxyglutarate. They can therefore be used to prevent and/or treat neurodegenerative diseases and cancers.

In Vitro Demonstration of the Antiproliferative Effect on Tumor Cells, in Particular HCT-116 Cells (Adenocarcinoma of the Colon).

The objective of this study is to demonstrate in vitro the effect of bacteria according to the invention, in particular of C. minuta on the proliferation of tumor cells. The effect of the supernatants was evaluated after 24 h and 48 h of treatment of a human colon adenocarcinoma cell line, the HCT-116 line. The procedure is described below.

Cell Culture

The HCT-116 cell line was maintained in culture in McCoy's 5A medium supplemented with 10% fetal calf serum (Gibco) and 1% penicillin/streptomycin (Sigma-Aldrich) in a humid incubator at 37° C. and 5% CO2.

Cell Treatment

The HCT-116 cells were seeded at a density of 10,000 cells/well in a total volume of 100 μl in a 96-well plate. After 24 h of incubation, the culture medium was removed from the adherent cells and new medium supplemented with 10% stationary phase supernatant of C. minuta was added (DSMZ: DSM22607, C. minuta 1, C. minuta 2 or C. minuta 3 and in GAM control (bacterial culture medium)). Each condition was done in 4 replicates. The cells were incubated for an additional 24 or 48 hours.

Proliferation Test

Cell proliferation was determined using the CellTiter-Glo 2.0 assay kit (Promega). The measurements were carried out according to the manufacturer's instructions. Briefly, the plates were removed from the incubator and allowed to equilibrate at room temperature for 30 min, and an equal volume of CellTiter-Glo 2.0 reagent was added directly to the wells (100 μl). The plates were stirred at 300 rpm for 2 min using a rotary stirrer, then incubated at room temperature for 10 min. The reaction mixture was then transferred to a white-walled 96-well plate and the luminescent signal was measured using a microplate reader (FLUOstar Omega, BMG Labtech).

Results

The supernatants of the different bacterial strains of the species C. minuta DSMZ: DSM22607, C. minuta 1, C. minuta 2 and C. minuta 3 significantly reduce the proliferation of cells treated for 24 h and 48 h, compared to the “GAM” control, corresponding to the cells treated with the bacterial culture medium (GAM vs DSMZ: DSM22607, C. minuta 2, C. minuta 2 p<0.0001, GAM vs C. minuta 1 p=0.0002 at 24 h and GAM vs DSMZ: DSM22607 p=0.0002, GAM vs C. minuta 1 p=0.0016, GAM vs C. minuta 2 and C. minuta 3 p<0.0001 at 48 h, Dunnett's multiple comparisons test). Akt Inhibitor VIII is used as a positive control for blocking cell proliferation.

Thus, the supernatants of the various tested bacteria according to the invention induce a reduction in the proliferation of tumor line HCT-116, making it possible to indicate a beneficial effect of the bacteria according to the invention in the inhibition of tumor development.

In Vitro Demonstration of the Antiproliferative Effect on Tumor Cells, in Particular HepG2 Cells (Hepatocarcinoma).

The objective of this study is to demonstrate in vitro the effect of bacteria according to the invention, in particular of C. minuta on the proliferation of tumor cells. The effect of the supernatants was evaluated after 24 h and 48 h of treatment of a human adenocarcinoma cell line, the HepG2 line. The procedure is described below.

Cell Culture

The HepG2 cell line was maintained in culture in DMEM medium supplemented with 10% fetal calf serum (Gibco) and 1% penicillin/streptomycin (Sigma-Aldrich) in a humid incubator at 37° C. and 5% CO2.

The cell treatment and the proliferation test are identical to the test previously described on the HCT-116 line.

### RESULTS

The supernatants of the bacteria according to the invention significantly reduce the proliferation of the cells treated at 24 h and all the strains of C. minuta DSMZ: DSM22607, C. minuta 1, C. minuta 2 and C. minuta 3 at 48 h, compared to the “GAM” control, corresponding to the cells treated with the bacterial culture medium (GAM vs DSMZ: DSM22607 or C. minuta 3, p=0.0003 and GAM vs C. minuta 2 p=0.0008 at 24 h/GAM vs DSMZ: DSM22607, C. minuta 2, C. minuta 3 p<0.0001 and GAM vs C. minuta 1 p=0.0024 at 48 h, Dunnett's multiple comparisons test). Akt Inhibitor VIII is used as a control for blocking cell proliferation.

Thus, the supernatants of the tested bacteria according to the invention induce a reduction in the proliferation of tumor line HepG2, making it possible to indicate a beneficial effect of the bacteria according to the invention in the inhibition of tumor development.

1. A human intestinal microbiota bacterium selected from bacteria of the Christensenellaceae family, bacteria of the Negativibacillus genus and bacteria of the Massiliomicrobiota genus, for its use in the prevention and/or treatment of at least one disease characterized by excess 2-hydroxyglutarate in humans or animals.

2. The bacterium for its use according to claim 1, in the prevention and/or the treatment of at least one neurodegenerative disease and/or of at least one cancer in humans or animals.

3. The bacterium for its use according to claim 1, in the prevention and/or treatment of at least one neurodegenerative disease, chosen from Alzheimer's disease, dementia, Parkinson's disease and related disorders, prion diseases, neuromuscular diseases, Huntington's disease, spinocerebellar ataxia, progressive spinal muscular atrophy and amyotrophic lateral sclerosis.

4. Bacterium for its use according to claim 2, in the prevention and/or the treatment of at least one brain and/or kidney and/or liver and/or colon cancer, in humans or animals.

5. Bacterium for its use according to claim 1, characterized in that said bacterium is a bacterium of the Christensenella genus.

6. Bacterium for its use according to claim 1, characterized in that said bacterium is selected from Christensenella massiliensis, Christensenella timonensis and Christensenella minuta, Negativibacillus massiliensis, and Massiliomicrobiota timonensis.

7. A composition comprising at least one human intestinal microbiota bacterium selected from bacteria of the Christensenellaceae family, bacteria of the Negativibacillus genus and bacteria of the Massiliomicrobiota genus for use in the prevention and/or treatment of diseases characterized by excess 2-hydroxyglutarate in humans or animals.

8. The composition for its use according to claim 7, in the prevention and/or treatment of neurodegenerative diseases and/or of at least one cancer in humans or animals.

9. The composition for its use according to claim 8, in the prevention and/or treatment of at least one neurodegenerative disease, chosen from Alzheimer's disease, dementia, Parkinson's disease and related disorders, prion diseases, neuromuscular diseases, Huntington's disease, spinocerebellar ataxia, progressive spinal muscular atrophy and amyotrophic lateral sclerosis.

10. The composition for its use according to claim 8, in the prevention and/or the treatment of at least one brain and/or kidney and/or liver and/or colon cancer, in humans or animals.

11. The composition for its use according to claim 7, characterized in that it also comprises at least one bacterium of the Christensenella genus.

12. The composition for its use according to claim 7, characterized in that it comprises at least one bacterium chosen from Christensenella massiliensis, Christensenella timonensis and Christensenella minuta, Negativibacillus massiliensis and Massiliomicrobiota timonensis, or a mixture of at least two of these bacteria.

13. The composition for its use according to claim 7, characterized in that it is in liquid form.

14. The composition for its use according to claim 7, characterized in that it is in solid form.

15. The composition for its use according to claim 14, characterized in that the bacteria are present in freeze-dried form.

16. The composition for its use according to claim 7, characterized in that the bacteria present are at least 50% living bacteria (by number).

17. The composition for its use according to claim 7, characterized in that the bacteria present are at least 90% living bacteria (by number).

18. The composition for its use according to claim 7, orally, rectally, inhaled.

19. The composition for its use according to claim 7, characterized in that it is in the form of powder, microencapsulated powder, gelcap, capsule, tablet, lozenge, granules, emulsion, suspension or suppository.

20. The composition for its use according to claim 7, characterized in that it is in a gastro-resistant form.

21. The composition for its use according to claim 7, characterized in that it comprises at least one probiotic and/or at least one prebiotic.

22. The composition for its use according to claim 7, characterized in that it also comprises:
at least one polyphenol, and/or
at least one mineral and/or at least one vitamin and/or at least one nutritional agent, and/or
at least one active pharmaceutical ingredient.
